(19) United States
(12) Patent Application Publication
(10) Pub. No.: US 2003/0092199 A1
(43) Pub. Date: May 15, 2003

(54) Prion-detection business methods

(76) Inventor: Kiamars Hajizadeh, Buffalo Grove, IL (US)

Correspondence Address: Wallenstein & Wagner, Ltd., 53rd Floor, 311 S. Wacker Drive, Chicago, IL, 60606-6622, US

(21) Appl. No.: 09/990,773

(22) Filed: Nov. 14, 2001

(51) Int. Cl. G01N 33/53; G01N 33/567; G01N 33/543

(52) U.S. Cl. 436/518; 435/7.1

(57) ABSTRACT

Methods are provided for rapid detection with high specificity of the pathogenic form of prion protein responsible for neurodegenerative diseases affecting humans and animals, such as transmissible spongiform encephalopathy in bovine, sheep, and cats. Methods are also provided for testing animal feedstock for pathogenic prio protein. Results are available in from about 0.5 to about 20 minutes and preferably within from about 5 to about 10 minutes. The methods employ proteinase-K to remove normal prion protein from a biological sample, so that the sample may be analyzed by immunochromatography to determine the presence and concentration of pathogenic prion protein. Because the proteinase-K is immobilized on a solid support for in-situ removal of interfering components, the present invention obviates the need for subsequent extraction of the desired analyte. All aspects of the present invention are suitable for quantifying the minimal detectable amount of pathogenic prion protein in a test sample. Moreover, the simplicity of sample preparation makes the present invention suitable for use in the field.

### TECHNICAL FIELD

This invention relates to rapid diagnostic methods for testing for disease in animals and humans, and more particularly to methods for detecting the pathogenic form of prion in animal feedstock and in biological fluids and tissues obtained from animals and humans suspected of having a prion-caused disease. The invention also relates to methods of testing animal carcasses for disease prior to processing and methods of testing animal feed made from animal parts for infectious prion protein.

### BACKGROUND OF INVENTION

Humans and animals develop a variety of transmissible neurodegenerative disorders as a result of infection by prions—aberrant proteins that join bacteria, viruses, and viroids as infectious pathogens. Examples of prion diseases afflicting animals include scrapie in sheep and goats, and bovine spongiform encephalopathy (BSE) in cattle. Animals may contract a prion disease by consuming feed made from organs and other components from infected animals, such as cow udders and bone in the form of bone meal. Humans are subject to four prion diseases including kuru, Creutzfeldt-Jakob disease, Gerstmann-Strassler-Scheinker disease, and fatal familial insomnia. Humans may contract Creutzfeldt-Jakob disease by consuming beef, as an example, infected with prions.

A conformational change that occurs in the normal host prion protein causes prion diseases by converting the normal prion protein into an abnormal aggregate-forming pathogenic structure known as a prion. The pathogenic form of prion protein is designated as “PrP^{SC}”; the normal form is designated as “PrP^{C}.”

Detection of prions is difficult because of the poor solubility of prions in many biological buffers and the tenacity of its aggregates in resisting dissolution. As a result, the methodology used for analyzing prions is oftentimes time-intensive and complex. For example, hydrophilic-interaction chromatography has been used to purify the abnormal prion protein, followed by capillary electrophoresis immunoassay for detection. Schmerr and Jenny, Electrophoresis 19:409 (1998), cited in U.S. Pat. No. 6,150,172.

Despite these problems, however, various assays are known in the art for selectively detecting abnormal prion protein Among the immunoassays for determining prion protein are techniques such as radioimmunoassay, ELISA (enzyme-linked immunosorbant assay), immunoradiometric assays, gel diffusion precipitation reactions, immunodiffusion assays, in situ immunoassays (using colloidal gold, enzyme or radioisotope labels), Western blots, precipitation reactions, agglutination assays (e.g., gel agglutination assays and hemagglutination assays), complement fixation assays, immunofluorescence assays, protein A and protein G assays, and immunoelectrophoresis assays.

Immunochromatographic assays are known for their ability to analyze proteins. For example, U.S. Pat. No. 6,180,417, issued to Hajizadeh et al., discloses an immunochromatographic assay, featuring both “sandwich” and competitive formats. U.S. Pat. Nos. 4,703,017 issued to Campbell et al. and 5,591,645 issued to Rosentein use visible particles in immunochromatography test strips. The test strip and assay of these patents, however, do not provide for the extraction and rapid analysis of pathogenic prion protein.

In U.S. Pat. No. 6,214,565, Prusiner et al. disclose a time- and labor-intensive assay for isolating and detecting the infectious prion protein in materials from human, bovine, sheep, goat and other animals. The assay involves treating a homogenized sample with a protease to remove substantially all non-infectious prion protein. The prion in the treated sample is then crosslinked to a plastic support. The filter is next immersed and incubated in an antibody-containing solution, followed by removal of the unbound antibody. The immersion/incubation/antibody-removal step is repeated with a second solution containing an anti-Ig antibody, typically radiolabled. Results are determined by immunoblot detection, using X-ray film. Conservatively, the assay takes at least four hours to prepare the filter for immunoblot detection.

U.S. Pat. No. 6,150,172 issued to Schmerr et al. discloses a three-step method for extracting abnormal prion protein from homogenized biological material and analyzing the extracted protein with a chromatographic immunoassay. The extraction method includes incubating an aqueous preparation of the biological sample with a pre-measured amount of proteinase-K to digest the normal prion protein, isolating the pathogenic prion protein by mixing the pre-treated sample with an extraction solvent, and recovering the isolated pathogenic prion protein in the extraction solvent. Col. 4, lines 21-26. The method shortens the extraction time to 1 to 2 hours. Col. 9, lines 27-28.

Schmerr et al. disclose that the extraction solvent can then be applied directly to a support and assayed via immunochromatography. The following U.S. patents set forth examples of immunochromatographic assays, known in the art that may be used for assaying the extraction solvent: U.S. Pat. Nos. 5,248,619; 5,451,504; 5,500,375; 5,624,809; and 5,658,801. Though the referenced method isolates and detects abnormal prion protein, it involves multiple steps and requires as much as two hours for merely extracting the analyte.

Thus, there exists a need for a device and simplified method for rapidly determining the presence and/or concentration of pathogenic prions in biological samples and animal feed. There also exists a need for test devices and assays that are capable of detecting nanogram quantities of pathogenic prion proteins, particularly, for example, for detecting prion diseases in medical applications and bovine spongiform encephalopathy in animal carcasses in the meat-processing industry.

### SUMMARY OF THE INVENTION

The present invention is directed to methods for determining the presence and concentration of pathogenic prion protein in animal feedstock and in biological samples obtained from a human or an animal. In each aspect of the invention, the pathogenic form of prion protein is readily extracted, essentially free of the normal nonpathogenic form of prion protein, and analyzed by immunochromatography.

A first aspect in accordance with the invention is a method for detecting disease in animal carcasses. The method begins with terminating an animal, followed by removing a biological sample from the terminated animal. The sample is homogenized with an analyte-extracting buffer to form a homogenate. The homogenate is treated with immobilized proteinase-K to remove interfering constituents. The enzyme-treated homogenate is then assayed for an analyte indicative of the disease by using a pair of antibodies specific to the analyte. A test result is obtained for the analyte in the sample and correlated to the animal so the carcass having a positive or negative test result may be removed.

The method further comprises, prior to the step of obtaining the biological sample, attaching a result display unit to the animal, where the result display unit is securely affixable to at least a portion of the test device. That portion of the test device indicates the positive result. In an alternative embodiment, the method further comprises processing nondiseased animals for use as food for humans and as ingredients for animal feed.

A second aspect of the invention is a method for diagnosing prion diseases in humans and animals. The method, having potential application in the medical community, comprises providing a biological sample from a vertebrate. The sample is homogenized with a buffer to form a homogenate containing extracted prion protein. The homogenized sample is introduced into a lateral flow device having immobilized proteinase-K for in situ digestion of interfering constituents and a pair of antibodies specific to the prion protein analyte for binding to the analyte. A test result is obtained for the prion protein analyte and correlated to the vertebrate from whom the biological sample was obtained.

A third aspect is directed to a method of detecting or measuring the concentration of infectious prion protein in foodstuffs such as animal feed or meat designated for human consumption, as examples. The method comprises providing a sample of a foodstuff and homogenizing the sample with a buffer to form a homogenate containing extracted prion protein. The homogenate is treated with immobilized proteinase-K to remove interfering constituents and the enzyme-treated homogenate is then assayed for a prion protein analyte indicative of a prion disease by using a pair of antibodies specific to the protein analyte. A test result, obtained from the assay, is correlated to the foodstuff for appropriate treatment thereof.

In all aspects of the invention, the test result is produced within from about 0.5 to about 20 minutes from the time the assaying step is started and preferably within about 5 to about 10 minutes. The assay has application in analyzing prion protein responsible for a number of prion-caused diseases in both animals and humans, such as transmissible spongiform encephalopathy (TSE) in bovine, sheep, and goats and Creutzfeldt-Jakob-disease (CJD) in humans. Because of the simplicity of sample preparation and analysis, the assay is especially suitable for use in the field; e.g., in both industrial meat processing and medical applications.

### BRIEF DESCRIPTION OF THE DRAWINGS

To understand the present invention, it will now be described by way of example, with reference to the accompanying drawings in which:

FIG. 1 is a side perspective view of one embodiment of a test device in accordance with the teachings of the present invention;

FIG. 2 is a side perspective view of another embodiment of a test device in accordance with the invention;

FIG. 3 is a top schematic view of another embodiment of a test device made in accordance with one aspect of the invention; and,

FIG. 4 is a side perspective view of still another embodiment of the test device made in accordance with the invention.

### DETAILED DESCRIPTION OF THE INVENTION

While this invention is susceptible of embodiments in many different forms, preferred embodiments of the invention are illustrated in the drawings and described in detail herein, with the understanding that the present disclosure is to be considered as an exemplification of the principles of the invention and is not intended to limit the broad aspect of the invention to the embodiments illustrated.

The present invention is directed to testing devices, systems, and methods that utilize immunochromatography for determining the presence and concentration of pathogenic prion protein in a biological sample. The present invention utilizes immobilized proteinase-K (PK) enzyme for in-situ removal of interfering components. The devices, systems, and methods are suitable for quantifying the minimal detectable amount of pathogenic prion protein in a biological sample. Moreover, the rapid detection of pathogenic prion protein with high specificity, combined with the simplicity of preparing the sample, makes the present invention suitable for use in the field.

The test devices, systems, and methods may be used for rapid detection of prion diseases such as scrapie and spongiform encephalopathy in bovine, sheep, cats, and other animals. Additionally, the devices, systems, and methods may be used by the medical community for analysis of human tissue for kuru, Creutzfeldt-Jakob disease, Gerstmann-Straussler-Scheinker disease and fatal familial insomnia.

Throughout this application, the following terms have the meanings set forth below.

“Biological material” or “biological sample” refers to fluid or tissue extracted from vertebrates, such as brain tissue, whole blood, serum, plasma, saliva, urine, and cerebral spinal fluid.

“Label” refers to a component or “tag” that is attached covalently to a protein of choice. The label could be from a number of detectable groups such as enzymes, visible particles, nanoparticles, and fluorescent components, as examples.

“PrP^{C}” refers to the nonpathogenic form of prion protein, which is enzymatically removed from the biological sample.

“PrP^{SC}” refers to the infectious or pathogenic prion protein which is the analyte in the methods of this invention.

Sample Preparation

The present methods, test devices, and systems are used to test biological material extracted from an animal or human. Samples of brain tissue, including organs, are extracted post-mortem; but other samples—such as urine, whole blood, serum, and plasma—may be obtained from the live animal or human. Alternatively, the sample may include, e.g., animal feed such as items traditionally made with animal parts such as bovine udders, bone, and other organs.

The biological sample is homogenized with a suitable quantity of buffer formulated to optimize the extraction of prion protein into the buffer medium. Homogenization may be accomplished by any technique known in the art, including, e.g., shaking the biological material with weights, vortexing the material, ultrasonic digestion, or comminuting the sample in a homogenizer. Preferably, however, homogenization is conducted by either vortexing or shaking the material with weights.

The buffer does not have organic solvents. Typically, the buffer is an aqueous solution formulated to have an ionic strength of from about 200 to about 400 mM to facilitate prion extraction from the sample. The buffer comprises at least one emulsifier or surfactant, casein, at least one polysaccharide such as a sugar, albumin such as bovine serum albumin (BSA), and a sufficient quantity of water to form a mixture. Typically, the emulsifiers include at least one emulsifier or surfactant such as octoxynol (e.g., IGEPAL®), nonoxynol, polyglycol ether (e.g., Tergitol® NP), polyoxythylene (10) isooctylphenyl ether, sodium dodecyl sulfate (SDS), or sodium deoxycholate, as examples. A preservative may be used; e.g., ethylene-diamine-tetraacetic acid (EDTA) and sodium azide. The polysaccharides include at least one of sucrose, mannose, trehalose, maltose, and other suitable polysaccharides, as examples, in an amount sufficient to yield a molar concentration ranging from about 60 to about 80 mM. Additionally, the buffer may contain a denaturing compound such as guanidine hydrochloride, urea, and guanidine isothiocyanate. The buffer may also contain a zwitterionic buffering salt, such as 4-(2-hydroxyethyl)-1-piperazineethane-sulfonic acid (HEPES), used at a concentration ranging from about 1.5 to about 5%, by weight, to maintain the integrity of the solid support for the enzyme used downstream in the analysis

The total concentration of the emulsifiers and surfactants ranges from about 0.05 to about 5%, by weight of the buffer, and the casein generally ranges from about 10 to about 40%, by weight of the buffer. The total concentration of the polysacccharides ranges from about 0.1 to about 30%, by weight of the buffer. The albumin is typically used at a concentration ranging from about 0.5 to about 4%, by weight of the buffer. The zwitterionic buffering agent may used at a concentration ranging from about 2 to about 5%, by weight. The denaturing agent may be present at a concentration ranging from about 0.1 to about 1 M.

An example of a suitable buffer is shown in Table 1.

TABLE 1
Example of a Buffer Formulation for Extracting Prion Protein.

| Buffer constituent | Concentration (wt %) |
|---|---|
| octoxynol | 0.1 |
| casein | 40.0 |
| HEPES | 3.0 |
| EDTA | 0.2 |
| trehalose | 0.1 |
| sucrose | 18.5 |
| BSA | 1.0 |
| NaCl | 1.5 |
| sodium deoxycholate | 0.5 |
| SDS | 0.4 |
| water | 34.7 |

The homogenate is prepared by homogenizing the biological sample with buffer in a weight/volume ratio of sample (mg) to buffer (ml) ranging from about 2:1000 to about 200:1000, and preferably from about 5:1000 to about 100:1000. Most preferably, the ratio of sample (mg) to buffer (ml) is about 30:1000 to about 70:1000.

A. The Test Device

Dry Device

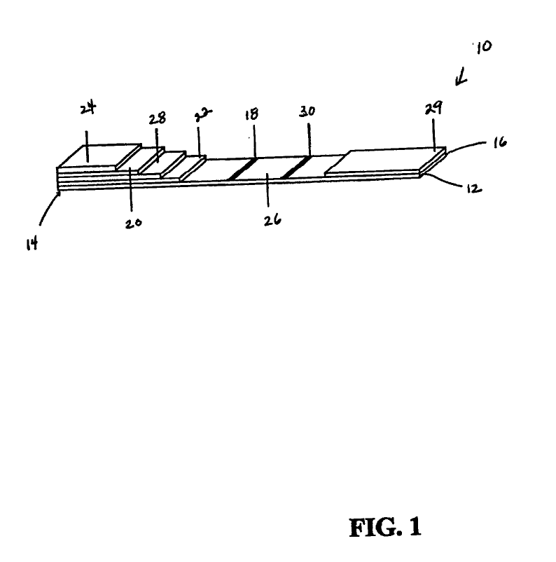

Shown in FIG. 1 is a test device 10 of a first embodiment. The test device 10 utilizes a pair of antibodies specific to PrP^{SC}. These include (1) a labeled antibody that “detects” the PrP^{SC} and (2) an immobilized antibody that “captures” the prion protein-antibody-label complex to form a “sandwich.” Briefly, in this invention, homogenized sample of a biological material is introduced to the test device. In the preferred embodiment, the sample first moves through a zone containing immobilized proteinase-K (PK), which digests the nonpathogenic prion protein, leaving the PrP^{SC} for analysis. The proteinase-K is immobilized to a solid support. The removal of the normal prion protein minimizes sample interference and results in a higher specificity for the analyte. As the treated sample moves through the test device, it encounters the first specific antibody conjugated to a label and affixed to a portion of the test device. In one embodiment, the label is a colored latex bead.

The fluid in the homogenized sample re-suspends the antibody-label conjugate so it is free to move through the device. As the antibody-label conjugate moves through the membrane, the labeled antibody binds to a particular epitope of the PrP^{SC} to form a prion protein-antibody-label complex. Via capillary force, the labeled complex migrates through the porous membrane of the device until it reaches the second specific antibody. This antibody is immobilized on the membrane, typically in the form of a band or stripe. The second antibody binds to the second epitope of the PrP^{SC} to which it is specific, resulting in the analyte becoming “sandwiched” between the two antibodies. The resulting “sandwiched” PrP^{SC} produces a detectable change in the membrane, such as the formation of a colored test line, which indicates a positive result. In the absence of antigen, no “sandwich” complex forms and no test line appears.

In an alternative embodiment, the test strip may include more than one “capture” antibody, each applied in a separate test line with each test line being specific to a different prion disease, so that the test device may be used for screening purposes.

The test device 10 includes a test strip 12 having an anterior end 14, a distal end 16, and a “test line” 18 therebetween. The test strip 12 comprises an absorbent material having pores (not shown) ranging from about 10 to about 1000 microns, and preferably from about 10 to about 100 microns. The pores are generally of a size sufficient to allow the homogenized sample, including the re-suspended labeled antibody and conjugates formed by the labeled antibody binding with prion proteins, to migrate laterally through the test strip 12 toward the test line 18.

The test strip itself has at least one layer of absorbent material. Suitable materials include at least one of, e.g., nitrocellulose, cellulose, glass fiber, bonded glass fiber, polyesters, nylon, polyethylsulphone, and other materials having absorbent properties, all of which allow an aqueous sample applied at one end of the test strip to migrate to the opposite end by capillary action.

Although FIG. 1 shows the nitrocellulose membrane or test strip 12 as being rectangular in shape, the test strip, of course, may have virtually any shape that allows an analyte to travel from a point where the sample is introduced to a point where the analyte is detected. Accordingly, the test strip may be square, triangular, circular, or octagonal, or any other suitable shape.

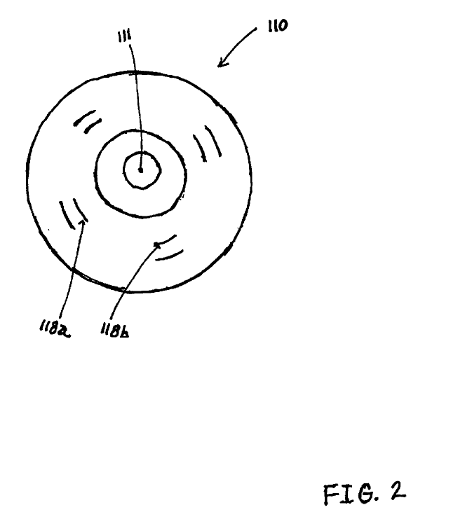

FIG. 2 shows the test device 110 having a circular configuration, with the immobilized antibody being affixed at a predetermined distance from the sample-introduction site 111. The embodiment shown in FIG. 2 has antibodies for two prion diseases and thus allows the respective pathogenic prion proteins to be analyzed for these in the same test device. Test lines 118a,b have immobilized antibodies corresponding to the pathogenic prion protein of a different prion disease which allows the device to be used as a diagnostic tool. Any of the test devices, irrespective of their shape, may be used to analyze more than prion disease at the same time.

In a preferred embodiment, the test strip 12 is affixed to a strip support 13 of a sufficiently rigid, impervious and non-reactive material such as polystyrene, polyvinyl chloride, and polyethylene terephthalates. Typically, the strip support is hydrophobic in nature to ensure that the maximum amount of test sample is directed for analysis. In a preferred embodiment, the strip support includes at least one layer of an impervious material.

In yet another embodiment, the entire test strip, and ancillary components described below, may be at least partially encased in a device holder for protecting the device from the environment. This form of the test device is best suited for use in more demanding test environments such as slaughterhouses.

At or near the anterior end 14 of the test strip 12, shown in FIG. 1, is a digestive pad 20 comprising immobilized proteinase-K for digesting nonpathogenic prion protein present in the homogenized biological sample. The digestive pad 20 is generally an absorbent material such as gauze but may comprise other suitable materials such as a plastic filter bed in glass fiber, polyester, and plastic bonded glass fiber, as examples.

The proteinase-K may be bound covalently to the digestive pad or conjugated to a solid support (not shown) impregnated in the digestive pad. The solid support may be, e.g., latex beads, rod-shaped bodies coated with latex, micro- or nanoparticles, beads coated with a dye or a fluorescent or chemiluminescent compound, or a porous membrane pad. Additionally, the proteinase-K may be incorporated into the digestive pad in a gelled substance contained therein. The latex beads in the digestive pad have an average diameter of from about 1 to about 10 microns.

The amount of enzyme on the support medium usually ranges from about 30 mg to about 400 mg and preferably from about 100 mg to about 350 mg. The amount of enzyme used should be sufficient to substantially digest all PrP^{C} present in the sample; typically, this amount is at least 30 units of enzyme per mg of all protein present in the sample. The enzyme treatment is conducted for a time and at a temperature sufficient for the proteinase-K to digest the nonpathogenic prion protein. Generally, digestion is completed in about 2 to about 15 minutes, depending upon the amount of prion present, when conducted at temperatures ranging from about 25° C. to about 60° C.

A conjugate pad 22 is disposed between the digestive pad 20 and the test strip 12, generally near the anterior end 14 of the test strip 12, and is impregnated with a label—typically a particulate—conjugated to one of the antibodies specific to the PrP^{SC}. As noted above, the particulates function as labels on the antibodies, allowing easy detection downstream on the nitrocellulose membrane. Suitable particulates for conjugation with the antibody include latex beads, rod-shaped bodies coated with latex, particles comprising a dye, colloidal particles, metal particles, micro- and nanoparticles, fluorescent compounds, chemiluminescent compounds, and magnetic beads, as examples. In one embodiment, the particulates are latex beads filled or coated with a dye, such as blue latex beads. The latex beads typically have an average diameter of from about 50 to about 500 nanometers and preferably from about 100 to about 350 nanometers. The magnetic beads have an average diameter of from about 50 to about 350 nanometers and preferably from about 100 to about 300 nanometers.

The conjugate pad comprises any absorbent material or suitable support for the labeled antibodies, such as a plastic filter bed in glass fiber, polyester, plastic bonded glass fiber, and other nonwoven materials, as examples. The conjugate pad lies in direct fluid communication with the test strip.

An alternative embodiment includes a filter pad 24 in fluid communication with the digestive pad 20, opposite the conjugate pad 22. Homogenized sample may be applied to the filter pad 24, an absorbent pad of a material that receives the fluid sample and allows it to flow into the conjugate pad 22. The filter pad 24 may also function to remove larger particles that may interfere with the assay. The filter pad 24 may comprise any suitable material such as gauze, cellulose, cellulose acetate, other polyesters, and other porous membranes, for example. Alternatively, the sample may be filtered in a separate step prior to its introduction to the digestive pad.

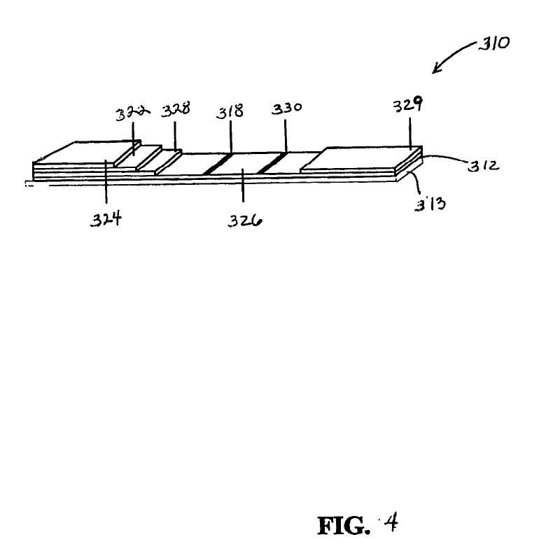

The test device 10 also has a detection region 26 (shown in FIG. 1 and designated by reference numeral “326” in FIG. 4) where the user may view the test result. The detection region 26 includes the test line 18 (shown as “318” in FIG. 4) and the control line 30 (shown as “330” in FIG. 4), when incorporated into the device.

As shown in FIG. 1, the three pads may be layered one atop the other at or near the anterior end, such that the filter pad 24 is the pad farthest from the test strip 12, the conjugate pad 22 is adjacent and substantially aligned with the test strip 12, and the digestive pad 20 is between the filter pad and the conjugate pad.

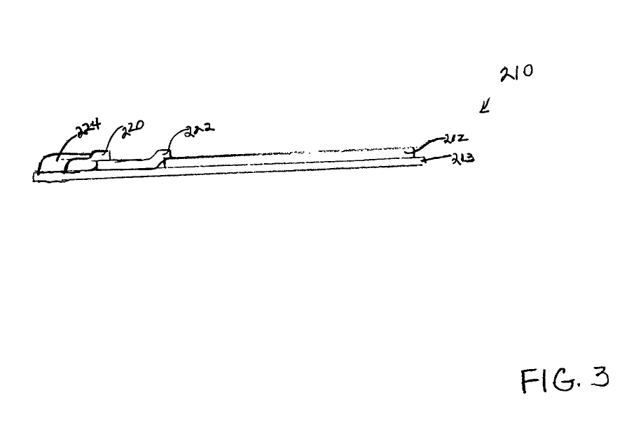

In a preferred embodiment of device 210, shown in FIG. 3, the pads lie substantially in the same plane, staggered with respect to each other, so that only a portion of one pad is in contact with a portion of an adjacent pad. Typically, the contact portion is in the form of an overlay between adjacent pads, such that the overlay between adjacent pads and between the test strip 212 and the adjacent pad ranges from about 0.5 to about 5 millimeters and preferably from about 1 to about 2 millimeters. Shown in FIG. 3 are filter pad 224, digestive pad 220, and conjugate pad 222. In the preferred embodiment, at least a portion of each pad and the test strip 212 is adhered to the support 213. The selection, shape, size, and positioning of the pads with respect to each other and the test strip 212 may be optimized as needed. In one embodiment, the pads may be distinct sections of one pad.

The order of the pads may be substantially as set forth above; e.g., the filter pad being the farthest from the detection region, followed by the digestive pad, and then, the conjugate pad. Each pad may have an outer edge generally corresponding in size and shape with that of the other pads, although other configurations are encompassed within the scope of this invention.

An additional pad may be needed to separate digestive pad from the conjugate pad. In another embodiment of the invention, the test strip may have a single pad impregnated with PK enzyme, serving both as the digestive pad and the filter pad. Though optional, a spacer pad 228 may be disposed between the digestive pad 220 and the conjugate pad 222 to allow for more complete digestion of the normal prion before it reaches the conjugate pad.

As shown in FIG. 1, in the detection region 26 lies the second antibody specific to the PrP^{SC}, typically immobilized on the membrane in the form of the “test line” or stripe. Alternatively, the antibody may be affixed in any suitable configuration that allows the test result to be viewed, or otherwise read, visually or by instrumentation. In another embodiment, the response may be compared against known responses or a standard curve to determine the concentration of the analyte.

In another embodiment, as shown in FIG. 1, the test device 10 includes a wicking pad 29 at the distal end of the test strip 14. The wicking pad 29 promotes the capillary flow of the homogenized fluid sample through the test strip by “drawing” the fluid sample to the distal end.

Generally, the amount of sample introduced to the test device is in the microliter range, typically from about 5 to about 500 microliters and preferably from about 75 to about 150 microliters.

In yet another embodiment, the test device includes a control line for indicating that the test is working properly. The control line, in fixed relation to the test line, comprises an antibody to the labeled antibody, such as immunoglobulin antibody, which binds with labeled antibody to produce a visually detectable line. Alternatively, the control line may be an antibody that binds with a secondary label on the particulate or bead, such as a protein or biotin-avidin binding sites.

The test line is permanent, but it could become visually more pronounced over time. Preferably, the test result is read within from about 2 to about 10 minutes from the time the homogenized sample is applied to the test strip.

The present invention allows pathogenic prion protein to be detected within from about 0.5 to about 20 minutes after sample is introduced to the test device and preferably within from about 5 to about 10 minutes. The invention allows substantially real-time reading of the results on the test strip so that a test result is available almost instantaneously. Therefore, the preferred embodiment of this invention employs enzyme digestion within the test device so that the sample is subjected to only one labor-intensive step; i.e., homogenization. However, when the enzyme pre-treatment is conducted separately from the test strip, detection via the immunochromatographic phase may be yield a readable result in from about 1 to about 5 minutes after sample introduction and preferably from about 2 to about 10 minutes, depending upon the concentration of normal prion protein to be denatured.

Device for Separate Enzyme Pretreatment

The PrP^{SC} may also be detected in biological samples and animal feeds by use of a testing system comprising (a) proteinase-K immobilized on a support external to the test strip, for digesting the nonpathogenic form of prion protein in a separate wet analysis conducted prior to introducing the homogenized sample to the test strip; and (b) a test strip that analyzes the enzymatically treated sample for the presence and concentration of PrP^{SC}. Shown in FIG. 4 is a test device 310, having an impervious strip support 313, that is suitable for use in this aspect of the invention. Test device 310 includes a conjugate pad 322, a detection region 326, and a test line 318. Optionally, the test device may also include one or more of a filter pad 324, a spacer pad 328, a control line 330, and a wicking pad 329. The test system is used with sample prepared as described above.

The test strip—including the antibodies, particulates, conjugate pad, and test line—and its operation are as described above for the device that performs both enzyme treatment and the assay. Additionally, the test strip or membrane may incorporate a control line, described above, for determining whether the test is operating correctly. In this aspect of the invention, the support having the immobilized enzyme separate from the test strip displaces the digestive pad.

This aspect of the invention has application, e.g., when the prion must be heated in order to be digested and the PK treatment cannot be performed in real time without heating.

This aspect of the invention includes several embodiments. In one embodiment, the support comprises magnetic beads. In an alternative embodiment, the support comprises, e.g., latex supports, filter tips, colloidal particles, microcrystalline particles, conjugate supports, plastic surfaces, and glass surfaces. The latex supports include, e.g., latex beads and latex-coated particles that may be of any shape. The amount of enzyme on the support medium ranges from about 30 micrograms to about 400 micrograms and preferably from about 100 micrograms to about 350 micrograms. The enzyme is used in an amount sufficient to substantially digest all PrP^{C} present in the sample; i.e., at least 30 units of enzyme per mg of all protein present in the sample.

When the sample is mixed with the support in, e.g., a test tube or a beaker, enzymatic digestion of the nonpathogenic prion protein is completed within about 15 minutes. Digestion is typically conducted at temperatures ranging from about 25° C. to about 60° C.

After digestion, the magnetic beads are separated from the mixture with a magnet rack or other suitable device, leaving a supernatant. Other forms of the solid support are removed from the treated sample by in-line filtration or any other suitable method. The supernatant is then applied to the test strip, without requiring further extraction of the prion analyte, for detecting and quantifying the PrP^{SC}. As described above, in the presence of PrP^{SC}, the test strip undergoes a detectable change, indicative of a positive result.

B. Assay

In another aspect of the invention, an assay is provided for detecting PrP^{SC} in a biological sample. The assay comprises homogenizing the sample with a suitable buffer, substantially as described above, and applying the homogenized sample to a test device or system, such as that described above and depicted in FIG. 1. The sample may be applied directly to the digestive pad or the filter pad, or it may be filtered onto either of such pads. Preferably, however, filtration is accomplished in situ directly by the device.

In the digestive pad, the homogenized sample is treated with the immobilized proteinase-K. As the homogenized sample and PrP^{SC} flow through the device, the antibody conjugated to a label, such as a colored bead or other particulate, binds the PrP^{SC} to form a labeled complex. By capillary force, the labeled antibody PrP^{SC} complex migrates through the detection zone membrane toward the immobilized antibody where it complexes with the immobilized antibody to produce a visually or otherwise readable response on the membrane, indicative of the presence or concentration of PrP^{SC}.

In still another embodiment, the assay comprises a two-step analysis wherein the buffered homogenized sample is first treated, in a separate wet chemical step, with proteinase-K immobilized on a support to obviate subsequent inactivation or removal of the enzyme. In this embodiment, the support is external to the lateral flow device and porous membrane. The treated sample is then applied to a lateral flow test device without the digestive pad, described above, for qualitative and quantitative analysis of PrP^{SC}.

The assay allows substantially real-time reading of the results on the test device so that results are available almost instantaneously. The enzymatic digestion of interfering constituents in situ requires the sample to be subjected to only homogenization prior to introduction to the device. However, when the enzyme pre-treatment is conducted separately from the test strip, detection via the immunochromatographic phase may be yield a readable result in from about 1 to about 5 minutes after sample introduction and preferably from about 2 to about 10 minutes, depending upon the concentration of normal prion protein to be denatured. Otherwise, results are available in from about 0.5 to about 20 minutes after the homogenate is introduced to the lateral flow device.

C. Methods

In yet another aspect of the invention, a method is provided for testing animal carcasses for disease and removing the diseased carcasses from further processing. In light of recent incidences of BSE, this aspect of the invention provides a rapid diagnostic method having enhanced sensitivity for identifying and removing diseased carcasses. The method may be used for detecting, e.g., spongiform encephalopathy in bovine, sheep, and goats and scrapie in sheep and goat. The method may also be used as a diagnostic tool for detecting transmissible mink encephalopathy (TME) in mink; chronic waste disease (CWD) in mule deer and elk; bovine spongiform encephalopathy (BSE) in cattle; feline spongiform in cats; and kuru, Creutzfeld-Jakob-disease (CJD), German-Straussler-Scheinker syndrome (GSS), and fatal familial insomnia (FFI) in humans.

In a first aspect of the invention, the method comprises terminating an animal and obtaining a sample of biological material, such as brain tissue, from the terminated animal. The sample is homogenized with a suitable buffer, as described above, to extract substantially all the prion protein into the homogenate so formed. The homogenate is then assayed for an analyte indicative of the disease being detected by using an immunochromatographic device, such as that described above, and a test result is obtained. The test result is correlated to the animal from which the sample was obtained so that the diseased carcass may be separated from the otherwise healthy ones. The assay may detect or quantify the analyte present in the sample.

In one embodiment of the invention, at least a portion of the test device may be attached to a part of the animal, before or after removing the biological sample from the animal, so as to ascribe or correlate the test result to the diseased or healthy animal. In another embodiment, a result display holder is attached to the animal prior to terminating the animal, so that the test device may be mated with the test device holder after the animal is terminated. The test device itself may be configured so the entire device or at least the portion displaying the test result can be mated to the device holder so the test result is readily apparent when viewing the animal.

The method also has application for testing humans for prion-related diseases. In a second aspect of the invention, a method for detecting prion diseases in humans and animals is provided. The method includes obtaining a biological sample from a human or an animal. This aspect of the invention is not invasive to the test subject, as the biological sample may be whole blood, serum, plasma, saliva, urine, and cerebral spinal fluid obtained from a living human or animal. Alternatively, tissue or fluid may be obtained from the carcass of an animal or the body of deceased human.

The biological sample is homogenized with a suitable buffer, such as that described above, to form a homogenate containing the extracted prion protein. The amount of biological sample used per 1000 ml is substantially as set forth above; i.e., the weight/volume ratio of sample (mg) to buffer (ml) ranges from about 2:1000 to about 200:1000, preferably from about 5:1000 to about 100:1000, and most preferably, from about 30:1000 to about 70:1000.

In another aspect of the invention, a method is provided for detecting or measuring the concentration of infectious prion protein in foodstuffs such as animal feed or meat designated for human consumption. The method begins with obtaining a representative sample of the foodstuff, typically by standard techniques known to those skilled in the art. The sample is homogenized with a buffer to extract the prion protein from the sample. In the homogenizing step, the foodstuff sample is typically used with the buffer in a weight(mg)/volume(ml) ratio ranging from about 5:1000 to about 400:1000 and preferably from about 10:1000 to about 200:1000.

In all aspects of the invention, the homogenate comprising either the biological sample or the animal feed or other foodstuff is then introduced into a lateral flow device having immobilized proteinase-K for in situ digestion of interfering constituents such as nonpathogenic prion protein and elimination of further sample-processing steps, which eliminates the need for subsequent extraction of the pathogenic prion analyte, followed by immunochromatographic analysis of the homogenate for the presence or concentration of pathogenic prion protein. The system utilizes an amount of proteinase-K sufficient to substantially, and preferably completely, digest the nonpathogenic prion protein. The amount of proteinase K immobilized in the device ranges from about 30 micrograms to about 400 micrograms, and preferably from about 100 micrograms to about 350 micrograms.

Both aspects of the invention may use either form of the test device or system—i.e., the device having an enzyme support external to the device or the system having the enzyme immobilized within a digestive pad, as described above.

As the homogenate flows through the test device, it re-suspends a labeled first antibody specific to the pathogenic prior protein. The membrane in the test device has pores of a diameter sufficient to allow the first labeled antibody to migrate laterally therethrough toward a second specific antibody immobilized in the system. A positive result occurs when the two antibodies, each specific for a different epitope of the pathogenic prion, bind with the prion. A test result for pathogenic prion protein is obtained and correlated to the source of the sample. The lateral flow device detects or quantifies the pathogenic prion protein in the sample.

The method yields a positive or negative result in from about 0.5 to about 20 minutes, and preferably within 15 minutes, after the homogenate is introduced to the lateral flow device to commence the assaying process. The lateral flow device is substantially as described above.

While the specific embodiments have been illustrated and described, numerous modifications come to mind without significantly departing from the spirit of the invention and the scope of protection is only limited by the scope of the accompanying claims.

We claim:

1. A method for detecting disease in animal carcasses comprising the steps of:
(a) terminating an animal;
(b) removing a biological sample from the terminated animal;
(c) homogenizing the sample with an analyte-extracting buffer to form a homogenate;
(d) treating the homogenate with immobilized proteinase-K to remove interfering constituents;
(e) assaying the enzyme-treated homogenate for an analyte indicative of the disease by using a pair of antibodies specific to the analyte;
(f) obtaining a test result for the analyte in the sample; and
(g) correlating the test result to the animal so the carcass having a positive or negative test result may be removed.

2. The method of claim 1 wherein the analyte causes transmissible spongiform encephalopathy.

3. The method of claim 1 wherein the test result is produced within from about 5 to about 10 minutes after commencing the assaying step.

4. The method of claim 1 wherein the homogenizing step comprises homogenizing the sample with a sufficient quantity of the buffer to extract the prion protein from the sample.

5. The method of claim 4 wherein the buffer is aqueous and comprises at least one emulsifier or surfactant, casein, at least one polysaccharide, and albumin.

6. The method of claim 5 wherein the at least one emulsifier or surfactant is selected from octoxynol, nonoxynol, polyglycol ether, polyoxythylene (10) isooctylphenyl ether, sodium dodecyl sulfate (SDS), and sodium deoxycholate.

7. The method of claim 5 wherein the at least one polysaccharide is selected from sucrose, mannose, trehalose, and maltose.

8. The method of claim 1 wherein the buffer has an ionic strength of from about 200 to about 400 mM.

9. The method of claim 1 wherein the assaying step is conducted in a test device comprising the immobilized proteinase-K and a lateral flow membrane for immunochromatographic analysis of the enzyme-treated homogenate.

10. The method of claim 9 wherein the step of correlating a test result to the animal includes attaching at least a portion of the test device to a part of the animal.

11. The method of claim 10 further comprising prior to removing the biological sample, attaching a test device holder to the animal for subsequent fastening thereto of the at least a portion the test device.

12. The method of claim 10 wherein the correlating step comprises separating the diseased carcass from nondiseased carcasses.

13. The method of claim 1 further comprising processing nondiseased animals for use as food for humans and as ingredients for animal feed.

14. A method for diagnosing prion diseases in humans or animals, comprising:
(a) obtaining a biological sample from a vertebrate;
(b) homogenizing the sample with a buffer to form a homogenate containing extracted prion protein;
(c) introducing the homogenized sample into a lateral flow device having immobilized proteinase-K for in situ digestion of interfering constituents and a pair of antibodies specific to the prion protein analyte for binding to the analyte;
(d) obtaining a test result for the prion protein analyte; and
(e) correlating the test result to the vertebrate from whom the biological sample was obtained.

15. The method of claim 14 wherein the pathogenic prion protein being analyzed causes a condition selected from the group consisting of spongiform encephalopathy in bovine, sheep, and goats and scrapie in sheep and goat; transmissible mink encephalopathy (TME) in mink; chronic waste disease (CWD) in mule deer and elk; bovine spongiform encephalopathy (BSE) in cattle; feline spongiform in cats; and kuru, Creutzfeldt-Jakob-disease (CJD), German-Straussler-Scheinker syndrome (GSS), and fatal familial insomnia (FFI) in humans.

16. The method of claim 14 wherein the biological sample is selected from blood, serum, plasma, saliva, urine, and cerebral spinal fluid.

17. The method of claim 14 wherein the biological sample is blood.

18. The method of claim 14 wherein the proteinase K is present in the test device in an amount ranging from about 30 micrograms to about 400 micrograms.

19. The method of claim 14 wherein the test result is obtained within from about 5 to about 10 minutes from the time of introducing the sample into the device.

20. The method of claim 14 wherein the homogenizing step comprises homogenizing the sample with a sufficient quantity of the buffer to extract substantially all the prion protein from the sample.

21. The method of claim 14 wherein the buffer comprises at least one emulsifier or surfactant, casein, at least one polysaccharide, albumin, and a sufficient quantity of water to form a mixture.

22. The method of claim 20 wherein the at least one emulsifier or surfactant is selected from octoxynol, nonoxynol, polyglycol ether, polyoxythylene (10) isooctylphenyl ether, sodium dodecyl sulfate (SDS), and sodium deoxycholate.

23. The method of claim 20 wherein the at least one polysaccharide is selected from sucrose, mannose, trehalose, and maltose.

24. The method of claim 14 wherein the buffer has an ionic strength of from about 200 to about 400 mM.

25. A method for detecting or measuring the concentration of infectious prion protein in foodstuff comprising the steps of:
(a) obtaining a sample of foodstuff;
(b) homogenizing the foodstuff with a buffer to form a homogenate;
(c) treating the homogenate with proteinase-K to digest nonpathogenic prion protein;
(d) assaying the enzyme-treated homogenate for a prion protein indicative of a prion disease by using an immunochromatographic technique;
(f) obtaining a test result from the assay; and
(g) correlating the test result to the animal feed.

26. The method of claim 25 wherein the prion protein being analyzed causes spongiform encephalopathy and Creutzfeld-Jakob-disease.

27. The method of claim 25 wherein the proteinase-K in the enzyme-treating step is immobilized on a support.

28. The method of claim 27 wherein the assaying step is conducted on a test device having
(a) a porous membrane through which the sample substantially free of nonpathogenic prion protein migrates by capillary action, the membrane being in fluid communication with the proteinase support; and
(b) a pair of antibodies specific to the pathogenic prion protein, one of the antibodies being immobilized on the membrane; and the other of the antibodies being labeled such that the labeled antibodies bind with the pathogenic prion protein and migrate toward the immobilized antibody.

29. The method of claim 25 wherein the proteinase-K is immobilized on a support selected from latex beads, rod-shaped bodies coated with latex, micro- or nanoparticles, and a porous membrane pad.

30. The method of claim 27 wherein the amount of proteinase K immobilized on the support is sufficient to substantially digest all protein in the sample.

31. The method of claim 30 wherein the amount of enzyme on the support ranges from about 30 micrograms to about 400 micrograms.

32. The method of claim 25 wherein the labeled antibody has a colored label.

33. The method of claim 27 wherein the buffer in the homogenizing step comprises at least one emulsifier or surfactant, casein, at least one sugar, salt, albumin, and a sufficient quantity of water to form a mixture.

34. The method of claim 25 wherein the homogenizing step comprises homogenizing the sample with the buffer in a weight(mg)/volume(ml) ratio ranging from about 5:1000 to about 400:1000.
